(19) United States
(12) Patent Application Publication
(10) Pub. No.: US 2006/0211628 A1
(43) Pub. Date: Sep. 21, 2006

(54) Treatment of multiple sclerosis with brain targeted anti oxidant compounds

(76) Inventors: Daphne Atlas, Jerusalem (IL); Eldad Melamed, Tel Aviv (IL); Daniel Offen, Kfar HaRoe (IL)

Correspondence Address: Martin Moynihan;Anthony Castorina, 2001 Jefferson Davis Highway, Suite 207, Arlington, VA, 22202, US

(21) Appl. No.: 10/522,766

(22) PCT Filed: Jul. 31, 2003
(86) PCT No.: PCT/IL03/00635

§ 371 Date: Feb. 27, 2006

Related U.S. Application Data
(60) Provisional application No. 60/400,114, filed on Aug. 2, 2002.

(51) Int. Cl. A61K 38/05; A61K 38/04; A61K 31/22; A61K 31/16; A61K 31/56

(52) U.S. Cl. 514/18; 514/19; 514/550; 514/625; 514/169

(57) ABSTRACT

A method of treating multiple sclerosis, the method comprises administering to a subject in need thereof a therapeutically effective amount of a compound, the compound having: (a) a combination of molecular weight and membrane miscibility properties for permitting the compound to cross the blood brain barrier of the organism; (b) a readily oxidizable chemical group for exerting antioxidation properties; and (c) a chemical make-up for permitting the compound or its intracellular derivative to accumulate within the cytoplasm of cells.

### FIELD OF THE INVENTION

The present invention relates, in general to the use of antioxidant compounds, also referred to herein as antioxidants, for the treatment of multiple sclerosis (MS). More particularly, the present invention relates to the use of brain targeted low molecular weight, hydrophobic antioxidants in the treatment of MS of any type and at any stage, including, for example, relapsing-remitting and chronic-progressive, either primary or secondary MS.

### BACKGROUND OF THE INVENTION

Multiple Sclerosis (MS) is a disorder of the central nervous system, involving decreased nerve function associated with the formation of scars on the myelin covering nerve cells. MS affects approximately 1 out of 1,600 people. 60% of MS patients are females. The disorder most commonly initiates between the ages of 20 to 40, and is one of the major causes of disability in adults under the age of 65.

Multiple sclerosis involves repeated episodes of inflammation of nervous tissue in various areas of the central nervous system, including the brain and the spinal cord. The location of the inflammation varies from one patient to another and from episode to episode of a given patient. The inflammation results in destruction of the myelin sheath covering the nerve cells in inflicted areas, causing the formation of multiple areas of scar tissue (sclerosis) along the covering of the nerve cells. Sclerosis slows or blocks the transmission of nerve impulses in that area, resulting in the appearance of the symptoms of MS.

MS symptoms vary considerably, since the location and extent of each attack varies. There is usually a stepwise progression of the disorder. At the initial stages (the “relapsing-remitting” stage) the episodes of onset of symptoms last days, weeks or months, alternating with times of reduced or no symptoms (remission) and periods of recurrence (relapse). During relapse there is an appearance of a new symptom, the reappearance of a previous symptom or the worsening of an existing symptom. At more advance stages of MS (termed: “chronic-progressive” stage, which may be either primary or secondary), there is a progressive deterioration of nerve function, which is probably caused by the irreversible destruction of nerve axons.

The exact cause of the inflammation associated with MS is unknown. Several geographic studies indicate that there may be an environmental factor involved with MS. There seems to be a familial tendency toward the disorder, with a higher incidence in certain family groups than in the general population, indicating a possible genetic involvement. An increase in the number of immune cells in the body of MS patients indicates that there may be a type of immune response that triggers the disorder.

The most frequent theories about the cause of multiple sclerosis include infection by a virus-type organism; abnormality of genes responsible for control of the immune system; or a combination of both factors.

There is no known cure for multiple sclerosis and current treatments are directed at reducing the symptoms of the disease in an attempt to provide MS patients with a better life quality.

MS medications vary depending on the symptoms that occur. Baclofen, dantroene, diazepam and other anti-spasmodic medications are used to reduce muscle spasticity. Cholinergic medications may be helpful to reduce urinary problems. Antidepressant medications may be helpful for mood or behavior symptoms. Amantadine may be administered for fatigue.

Corticosteroids or ACTH are frequently used to suppress the inflammation in an attempt to reduce the duration of an attack. Medications that suppress the immune system are also often used. Recently it has been found that Interferon may also be helpful for some patients.

Oxidative Stress and Various Neurodegenerative Pathologies

In the last few years evidences have accumulated which connect oxidative stress (OS) with the pathogenesis of Pakinson's, Alzheimer's, Creutzfeldt-Jakob's diseases and other human neurodegenerative disorders (Olanow, 1990, 1993; Fahn and Cohen, 1992; Butterfield and Lauderback., 2002, Brown et al., 1996; Thomas et al., 1996).

PCT/US97/23997 and corresponding patents and applications teach novel brain targeted low molecular weight, hydrophobic antioxidants and the use of such antioxidants in the treatment of central nervous system neurodegenerative disorders such as Parkinson's, Alzheimer's and Creutzfeldt-Jakob's diseases and amyotrophic lateral sclerosis and in treatment of conditions of peripheral tissues, such as acute respiratory distress syndrome, atherosclerotic cardiovascular disease and multiple organ dysfunction, in which oxidants are overproduced. PCT/US97/23997, however, fails to teach the use of such antioxidants for treatment of MS.

Experimental Animal Model of Multiple Sclerosis

An extremely useful animal model was established to help in understanding of the mechanism of the MS disease and to develop novel therapeutic strategies. The model is experimental autoimmune encephalomyelitis (EAE) with clinical signs and lesions that closely resembling those observed in MS (Martin, 1992). Several drugs were so far developed for MS, based on this animal model and are used for treatment of the disease.

Oxidative Stress and Multiple Sclerosis

Since inflammation is one of the first events during the demyelinating process in MS, free radicals may play a major role in the oligodendrocyts cell death and in the axonal damage. However, little is known about the role of antioxidants in MS. It has been shown that MS patients have significantly lower levels of serum uric acid (Hooper et al., 1998), plasma vitamin E, and ubiquinone, lymphocyte ubiquinone and erythrocyte glutathione peroxidase than controls (Syburra and Passi, 1999).

Natural antioxidants were suggested for the treatment of MS. Recently uric acid, a strong peroxynitrite scavenger, has been used successfully in treating the EAE animal model of MS (Hooper et al., 1998). Protection against the cytotoxic and DNA-damaging effects of NO were also demonstrate in an EAE model (Schwarz et al., 1995 and Tsangaris et al., 1998).

A further indication of the involvement of oxidative stress in MS stems from the observation that oxidative stress plays a role in the pathogenesis of EAE (Lin et al., 1993; Cross et al., (1994); Okuda et al., (1995); Ruuls et al., (1996); Fenyk et al., (1998) and Sahrbacher et al., 1998, Offen et al., 2000).

Another interesting link between MS and oxidative stress came from the study of metallothioneins (MTs), a family of low molecular weight, heavy metal-binding, cysteine-rich proteins. It has been demonstrated that MTs accumulate under conditions where oxidative stress has taken place (Shiraga et al., 1993) and they may provide protection against oxygen radicals and oxidative damage caused by inflammation, tissue injury and stress (Ebadi et al. 1995).

In a recent study it was demonstrated that EAE mice showed a significant induction of metallothioneins I and II in the spinal cord white matter, and to a lower extent in the brain. These results suggest that metallothioneins I and II play an important role during experimental autoimmune encephalomyelitis (Espejo et al., 2001). Previously it was demonstrated that MTs show cytoprotective effects that appear to be related to their ability to act as scavengers of oxygen free radicals, such as hydroxyl and superoxide radicals (Thomalley et al., (1985) and Lazo et al., (1995).

These studies indicate that the thiol-groups within the cysteine rich enzymes such as metallothioneins I and II could be a target for oxidation by the free radicals that are increased in oxidative stress conditions (Aschner 1997, Aschner et al., 1997).

### SUMMARY OF THE INVENTION

While conceiving the present invention, it was hypothesized that thiol-based brain targeted, low molecular weight, hydrophobic antioxidants that would effectively cross the blood brain barrier (BBB) and penetrate into the damaged brain tissue may help to maintain the redox status of the neurons, decrease ROS-associated neuronal damage and protect specific enzymes that protect the cells from inflammation. Even if the BBB is opened during lymphocytes penetration and/or the progression of the disease, supplementing the brain with an antioxidant that readily crosses the BBB would be helpful in the treatment of multiple sclerosis.

While reducing the present invention to practice it was shown that in an animal model of multiple sclerosis (MS) an experimental autoimmune encephalomyelitis (EAE) model produced by an injection of myelin oligodendrocyte glycoprotein (MOG), animals treated by the administration of a thiol-based brain targeted, low molecular weight, hydrophobic antioxidant before the appearance of clinical MS-related symptoms developed virtually no such clinical symptoms, and appeared essentially normal in all aspects evaluated.

According to one aspect of the present invention there is provided a method of treating multiple sclerosis, the method comprising administering to a subject in need thereof a therapeutically effective amount of a compound, the compound having: (a) a combination of molecular weight and membrane miscibility properties for permitting the compound to cross the blood brain barrier of the organism; (b) a readily oxidizable chemical group for exerting antioxidation properties; and (c) a chemical make-up for permitting the compound or its intracellular derivative to accumulate within the cytoplasm of cells.

According to another aspect of the present invention there is provided a method of therapeutically or prophylactically treating a subject against multiple sclerosis, the method comprising administering to the individual a therapeutically or prophylactically effective amount of an antioxidant compound, the antioxidant compound having: (a) a combination of molecular weight and membrane miscibility properties for permitting the compound to cross the blood brain barrier of the individual; (b) a readily oxidizable chemical group for exerting antioxidation properties; and (c) a chemical make-up for permitting the compound or its intracellular derivative to accumulate within brain cells of the individual.

According to yet another aspect of the present invention there is provided a pharmaceutical composition for therapeutically or prophylactically treating a subject against multiple sclerosis, the composition comprising a pharmaceutically acceptable carrier and, as an active ingredient, a therapeutically or prophylactically effective amount of an antioxidant compound, the compound having: (a) a combination of molecular weight and membrane miscibility properties for permitting the compound to cross the blood brain barrier of the individual; (b) a readily oxidizable chemical group for exerting antioxidation properties; and (c) a chemical make-up for permitting the compound or its intracellular derivative to accumulate within brain cells of the individual.

According to still further features in the described preferred embodiments the compound is selected from the group consisting of N-acetyl cysteine ethyl ester (compound A), β,β-dimethyl cysteine ethyl ester (compound B), N-acetyl-β,β-dimethyl cysteine (compound C), Glutathione ethyl ester (compound D), N-acetyl glutathione ethyl ester (compound E), N-acetyl glutathione (compound F), N-acetyl α-glutamyl ethyl ester cysteinyl glycyl ethyl ester (compound G) N-acetyl α-glutamyl ethyl ester cysteinyl glycyl (compound H), N-acetyl glutathione amide (compound I), N-acetyl cysteine amide (compound J), N-acetyl β,β dimethyl cysteine amide (compound K) and N-acetyl cysteine glycine amide (compound L).

According to still further features in the described preferred embodiments the readily oxidizable chemical group is a sulfhydryl group.

According to still further features in the described preferred embodiments the chemical make-up is selected having an ester moiety which is removable by hydrolysis imposed by intracellular esterases.

According to still further features in the described preferred embodiments the ester moiety is selected from the group consisting of alkyl ester and aryl ester.

According to still further features in the described preferred embodiments the alkyl and aryl esters are selected from the group consisting of methyl ester, ethyl ester, hydroxyethyl ester, t-butyl ester, cholesteryl ester, isopropyl ester and glyceryl ester.

According to still further features in the described preferred embodiments the pharmaceutically acceptable carrier is selected from the group consisting of a thickener, a buffer, a diluent, a surface active agent and a preservatives.

The present invention successfully addresses the shortcomings of the presently known configurations by providing a novel method and pharmaceutical composition for the therapeutic or prophylactic treatment of multiple sclerosis.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show details of the invention in more detail than is necessary for a fundamental understanding of the invention, the description taken with the drawings making apparent to those skilled in the art how the several forms of the invention may be embodied in practice.

In the drawings:

FIG. 1 presents [³H]-thymidine uptake by PC12 cells treated in vitro with 0.5 mM dopamine which confers extracellular oxidative stress by forming oxidation products during its oxidation in the medium rescued with various concentrations of compounds A-D;

FIG. 2 presents [³H]-thymidine uptake by PC12 cells treated in vitro with 0.5 mM 6-hydroxy-dopamine, which confers intracellular oxidative stress by first entering the cytoplasm and then forming oxidation products during its oxidation in the cytoplasm, protected with various concentrations of compounds A-D and exogenous reduced glutathione (GSH); and

FIG. 3 presents the ratio of endogenous reduced glutathione levels in striatum/serum in two mice injected with 100 mg/kg of compound A in 3% DMSO, 100 mg/kg of reduced glutathione in 3% DMSO, and 3% DMSO injected as a control group, wherein the ratio obtained is marked at the top of the columns. The results represent two animals where each striatum taken separately.

FIG. 4 demonstrates that Compound J at as low as 0.1 mM protect NB cells against the toxicity (>50%) of DA, L-dopa (levodopa), 6-OHDA (0.1-0.25 mM) and MPP⁺ (0.5-2 mM). Cell survival was monitored by the neutral red assays.

FIG. 5a shows HPLC profile of purified Compound J.

FIG. 5b shows HPLC profile of a brain extract of a mouse 15 minutes following IP injection of compound J.

FIG. 6 shows the concentration of compound J in brain extracts of mice 15 minutes following IP injection of compound J at the amounts indicated.

FIG. 7 shows the mean clinical score of an EAE model of MS (mice injected with MOG) as a function of time after injection, for untreated mice (full circles) and mice treated with Compound J (full triangles).

FIG. 8 shows the percentage of disease free MOG-induced EAE mice a function of time after injection, in the untreated group (full circles) and in mice treated with Compound J (full triangles).

### DESCRIPTION OF THE PREFERRED EMBODIMENTS

The present invention is of methods and pharmaceutical compositions for the treatment of multiple sclerosis. Specifically, treatment of multiple sclerosis according to the present invention comprises the use of low molecular weight, hydrophobic, brain targeted antioxidants.

The principles of operation of the methods and pharmaceutical compositions according to the present invention may be better understood with reference to the drawings and accompanying descriptions.

Before explaining at least one embodiment of the invention in detail, it is to be understood that the invention is not limited in its application to the details set forth in the following description or illustrated in the Examples section that follows. The invention is capable of other embodiments or of being practiced or carried out in various ways. Also, it is to be understood that the phraseology and terminology employed herein is for the purpose of description and should not be regarded as limiting.

The term “multiple sclerosis” refers to MS of any type, and at any stage including, for example, relapsing-remitting and chronic-progressive, and also to any other autoimmune disease manifested by demyelinating of the central nervous system's neurons.

The term “treatment” in the context of the invention refers to any one of the following: amelioration of some of the undesired symptoms of multiple sclerosis; the prevention of the manifestation of such symptoms before they occur; slowing down or completely preventing the progression of the disease (as may be evident by longer periods between reoccurrence episodes, slowing down or prevention of the deterioration of symptoms, etc.); enhancing the onset of a remission period; slowing down the irreversible damage caused in the progressive-chronic stage of the disease (both in the primary and secondary stages); delaying the onset of said progressive stage, or a combination of two or more of the above.

Antioxidant compounds are used according to the present invention to relieve oxidation stress within cells of at the CNS and peripheral cells, suffering from MS, MS according to the present invention may be due, even if in part, to an overproduction of reactive oxygen species (ROS), or reactive nitrogen species (RNS).

A compound which is used to relieve oxidation stress in the central nervous system of MS patients according to the present invention (i) has a combination of molecular weight and membrane miscibility properties rendering it capable of crossing the blood brain barrier; (ii) includes a readily oxidizable (i.e., reduced) chemical group, such as, but not limited to, a sulfhydryl (—SH) group, for exerting antioxidation properties; and (iii) has a chemical make-up for permitting it or its cellular derivative(s) to accumulate within the cytoplasm of cells, such as brain cells. Collectively, these properties render the compounds suitable for treatment of the CNS.

Compounds which have the above listed properties are for example:

(i) N-acetyl cysteine ethyl ester —C₇H₁₁NO₃S— of a formula (compound A)

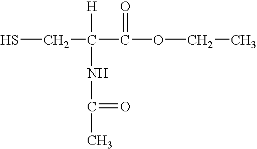

(ii) β,β-dimethyl cysteine ethyl ester or N-acetyl-penicillamine ethyl ester —C₉H₁₈NO₃S— of a formula (compound B):

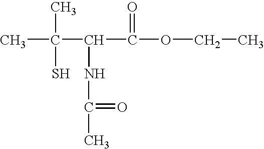

(iii) N-acetyl-β,β-dimethyl cysteine or N-acetyl-penicillamine —C₇H₁₃NO₃S— of a formula (compound C):

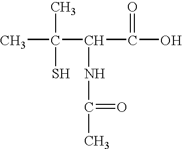

(iv) Glutathione ethyl ester —C₁₂H₂₁N₃O₆S— of a formula (compound D):

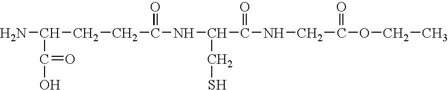

(v) N-acetyl glutathione ethyl ester —C₁₄H₂₃N₃O₇S— of a formula (compound E):

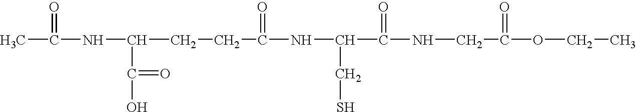

(vi) N-acetyl glutathione —C₁₂H₁₉N₃O₇S— of a formula (compound F):

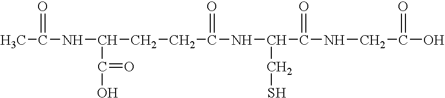

(vii) N-acetyl α-glutamyl ethyl ester cysteinyl glycyl ethyl ester or N-acetyl (α-ethyl ester) glutathione ethyl ester —C₁₆H₂₇N₃O₇S— of a formula (compound G):

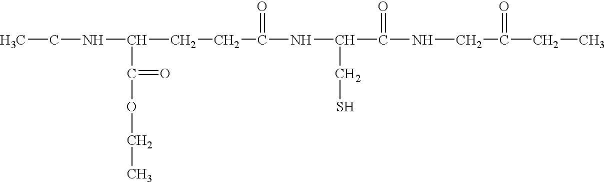

(viii) N-acetyl α-glutamyl ethyl ester cysteinyl glycyl or N-acetyl (α-ethyl ester) glutathione —C₁₄H₂₃N₃O₇S— of a formula (compound H):

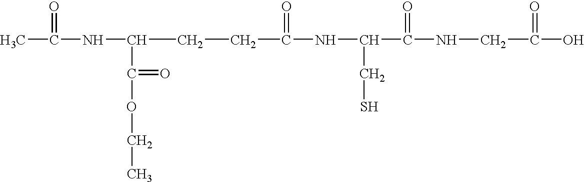

Additional compounds which may serve as antioxidants according to the present invention are:

(ix) N-acetyl glutathione amide —C₁₂H₂₁N₅O₅S— of a formula (compound I):

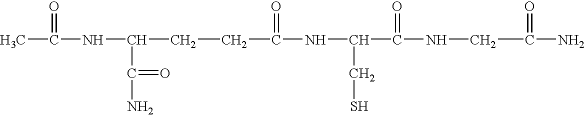

(x) N-acetyl cysteine amide —C₅H₁₀N₂O₂S— of a formula (compound J):

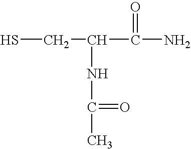

(xi) N-acetyl β,β dimethyl cysteine amide —C₇H₁₅N₂O₂S— of a formula (compound K):

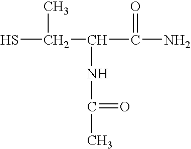

(xii) N-acetyl cysteine glycine amide —C₇H₁₂N₃O₃S— of a formula (compound L):

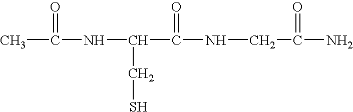

These compounds are used according to the present invention as antioxidants which cross the blood brain barrier, for relieving oxidative stress in cases of MS.

According to a preferred embodiment of the invention, the compound is a pro-drug, which penetrates the cells due to its solubility in the cell membrane and is hydrolyzed once inside the cell, exerting a drug having the antioxidant activity. For example compounds A, B, D, E, G and H above are pro-drug compounds.

Compounds A, B, E, G and H are pro-drug compounds, and their hydrolytic products ethanol and N-acetyl-cysteine (for compound A); ethanol and N-acetyl-penicillamine (for compound B); ethanol and N-acetyl glutathione (for compounds E, G and H) are known not to be toxic. The lethal dose 50% (LD50) value for N-acetyl-cysteine is 5,050 mg/kg. N-acetyl-penicillamine is available as an oral medication distributed under the generic name cuprimine by various manufacturers. Whereas N-acetyl glutathione and ethanol are both well known to be non-toxic substances.

A pro-drug according to the present invention includes at least one ester moiety such as an alkyl ester or an aryl ester, e.g., methyl ester, ethyl ester, hydroxyethyl ester, t-butyl ester, cholesteryl ester, isopropyl ester and glyceryl ester.

Preferably the pro-drug includes an ethyl ester moiety which, on one hand, neutralizes the charge of the carboxylic group(s) and on the other hand, when hydrolyzed within the cells release ethanol which is a substance known not to be toxic to the cells.

Upon entering the cytoplasm of a cell, the pro-drug is de-esterified by one or various intracellular esterases, to release the drug which has at least one carboxyl moiety (—COOH) and a by-product (typically ethanol) which contains the hydroxyl moiety (—OH). The carboxylic group(s) of the drug is typically negatively charged and the drug therefore is trapped within the cell, where it is to exert its antioxidative properties.

Compounds A and B are synthesized as follows: First, N-acetyl cysteine (for compound A) or N-acetyl β,β-dimethyl cysteine (for compound B) is mixed with a cooled solution of thionyl chloride and absolute ethanol. Second, the mixture is refluxed. And third, the volatiles are removed from the mixture for obtaining a first residue. Preferably the method further includes the following step. Fourth, the first residue is dissolved in water. And fifth, the first residue is extracted from the water with methylene chloride. Preferably the method further includes the following step. Sixth, the extract is dried to obtain a second residue. Preferably the method further includes the following step. Seventh, the second residue is crystallized from petroleum ether (for compound A) or from a methanol water solution (for compound B). Further detail concerning the method of preparing compounds A and B are delineated herein below in the Examples section.

Compound C is described in Biochem. Prep. 3, 111 (1953) and in U.S. Pat. Nos. 2,477,148 and 2,496,426, both are incorporated by reference as if fully set forth herein, and was prepared essentially as therein described. As mentioned above, compound C, N-acetyl-penicillamine, is available as an oral medication distributed under the generic name cuprimine by various manufacturers.

Compound D above is commercially available from Sigma Biochemicals, Cat. No. G1404. Compounds D is a pro-drug compound, and its hydrolytic products ethanol and glutathione are well known not to be toxic.

Compounds E, G and H above are glutathione derivatives and can be prepared, for example, from commercially available building units for Boc and Fmoc chemistry peptide synthesis, as well known in the art.

Compound F is a glutathione derivative and is described in Levy et al., 1993.

Compounds I, J and K are synthesized as follows. First, ammonia gas is bubbled through absolute cooled dry ethanol. Second, N-acetyl glutathione ethyl ester (compound G, for synthesis of compound I), N-acetyl cysteine ethyl ester (compound A, for synthesis of compound J) or N-acetyl β,β dimethyl cysteine ethyl ester (compound B, for synthesis of compound K) is added to the ethanol solution. Third, a container holding the reaction is sealed. Fourth, access ammonia and ethanol are evaporated and finally the resulting product is lyophilized. Further detail concerning the method of preparing compounds I, J and K are delineated herein below in the Examples section.

The synthesis of Compound L is further detailed in the Examples section that follows.

Any of the glutathione-derived compounds (D-H and I) may be prepared employing Boc and Fmoc chemistry for peptide synthesis. This, in turn, permits the inclusion of native Levo (L isomer) and/or non-native Dextro (D isomer) glutamic acid and/or cysteine derivatives or residues within any of these compounds. It will be appreciated that by replacing the native L configuration by the non-native D configuration, a compound becomes less recognizable by many enzymes and its biological half-life within the body therefore increases. Compounds A-C and J-K also include chiral carbons. Any of these carbons may also acquire a D or an L isomreric configuration.

Thus, compounds A-L above were given chemical names according to all L isomer configurations, i.e., all of their chiral carbon atoms are L isomers. However, as used herein in the specification and in the claims, these chemical names also refer to any of their D isomer(s) containing chiral atoms.

As mentioned above, compounds D-H are glutathione derivatives. These compounds and similar glutathione derivative compounds are represented by the general formula:

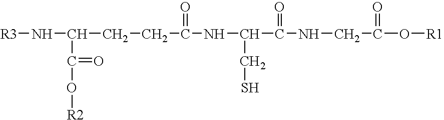

R1 is selected from the group consisting of a hydrogen atom and an alkyl (e.g., C₁-C₂₀) or aryl (e.g., C₆-C₉) group. Preferably R1 is an ethyl group.

R2 is selected from the group consisting of a hydrogen atom and an alkyl (e.g., C₁-C₂₀) or aryl (e.g., C₆-C₉) group. Preferably R2 is a ethyl group.

Whereas, R3 is selected from the group consisting of a hydrogen atom and an R4-CO (acyl) group, wherein R4 is an alkyl (e.g., C₁-C₂₀) or aryl (e.g., C₆-C₉) group. Preferably R4 is a methyl group. However, any one of R1, R2 and R4 can independently be a methyl, ethyl, hydroxyethyl, t-butyl, cholesteryl, isopropyl or glyceryl group.

Compounds A, B and E-L are not listed in the Chemical Abstract.

For therapeutic or prophylactic treatment of MS, the antioxidant compounds of the present invention can be formulated in a pharmaceutical composition.

Hence, further according to the present invention there is provided a pharmaceutical composition including one or more of the compounds described herein as active ingredients.

As used herein a “pharmaceutical composition” refers to a preparation of one or more of the antioxidant compounds described herein, with other chemical components such as pharmaceutically suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration of the pharmaceutical compositions of the invention may, for example, include oral, rectal, transmucosal, transdermal, intestinal or parenteral delivery, including intramuscular, subcutaneous and intramedullary injections as well as intrathecal, direct intraventricular, intravenous, intraperitoneal, intranasal, or intraocular injections.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer with or without organic solvents such as propylene glycol, polyethylene glycol.

For transmucosal administration, penetrants are used in the formulation. Such penetrants are generally known in the art.

For oral administration, the compounds can be formulated readily by combining the active antioxidant compounds with pharmaceutically acceptable carriers well known in the art. Such carriers enable the compounds to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the compounds to allow for the preparation of highly concentrated solutions.

Alternatively, the active ingredients may be in powder form for constitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of compound effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any compound used in the methods of the invention, the therapeutically effective amount or dose can be estimated initially from activity assays in animals. For example, a dose can be formulated in animal models to achieve a circulating concentration range that includes the IC₅₀ as determined by activity assays. Such information can be used to more accurately determine useful doses in humans.

Toxicity and therapeutic efficacy of the compounds described herein can be determined by standard pharmaceutical procedures in experimental animals, e.g., by determining the IC₅₀ and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See, e.g., Fingl et al., 1975, in “The Pharmacological Basis of Therapeutics”, Ch. 1 p. 1).

Dosage amount and interval may be adjusted individually to provide plasma levels of the active moiety which are sufficient to maintain the modulating effects, termed the minimal effective concentration (MEC). The MEC will vary for each preparation, but can be estimated from in vitro data. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

Depending on the severity and responsiveness of the condition to be treated, dosing can also be a single administration of a slow release composition described hereinabove, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition. Suitable conditions indicated on the label may include multiple sclerosis.

Hence, persons ordinarily skilled in the art can easily determine optimum dosages, dosing methodologies and repetition rates. Administration is preferably effected as soon inflammation or MS are diagnosed.

The compounds described herein for the treatment of MS are anticipated to synergize with presently known and to be developed other compounds effective in MS treatment, such as, but not limited to, copaxon, beta-interferons, Riluzole and IVIG (immunoglbulins).

It will be appreciated that the pharmaceutical compositions of the present invention are suitable to be administered to patients in all stages of the disease of multiple sclerosis, including the initial relapsing-remitting stages, both during remission periods (to prevent or delay reoccurrence) or reoccurrence conditions (to expedite remission and delay the onset of the progressive stage), as well as in chronic-progressive stages.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference in now made to the following examples, which together with the above descriptions, illustrate the invention.

### Example 1

### Synthesis of N-Acetyl Cysteine Ethyl Ester (Compound A)

N-acetyl cysteine (4.6 mmol) was added in portions to a cooled (e.g., 2-8° C.) solution of 2 ml thionyl chloride and 10 ml absolute ethanol. The resulting mixture was refluxed at 40° C. for 1 hour and then the volatiles were removed in vacuo. The residue was dissolved in 10 ml of water and was extracted twice with 20 ml of methylene chloride. The extract was dried under vacuo. The title compound was crystallized from petroleum ether (fraction 40-60°) in 55% yield.

The resulting product has the following characteristics:
(a) Melting point of 90° C.
(b) Anal. calculated for C₇H₁₁NO₃S:
   Calculated: C, 43.9; H, 6.8.
   Found: C, 42.5; H, 6.0.
(c) Thin layer chromatography in n-butanol/acetic acid/water (4/1/4) was carried out and the Rf value was Rf=0.91. The Rf value of the reactant, N-acetyl cysteine is 0.78.
(d) Nuclear Magnetic Resonance (NMR) in deutarated trichloromethane (CDCl3):
   6.51, 0.7H
   4.85, 1H, m
   4.23, 2H, q, J=7.0
   3.44, 0.4H, d, J=4.4
   3.22, 2H, t, J=4.4
   2.06, 3H, S
   1.30, 3H, t, J=7.0

### Example 2

### Synthesis of N-Acetyl β,β-Dimethyl Cysteine Ethyl Ester or N-Acetyl-Penicillamine Ethyl Ester (Compound B)

N-acetyl β,β-dimethyl cysteine (2.6 mmol) was added in portions to a cooled (2-8° C.) solution of 2 ml thionyl chloride and 10 ml absolute ethanol. The resulting mixture was refluxed at 40° C. for 1 hour and then the volatiles were removed in vacuo. The residue was dissolved in 10 ml of water and was extracted twice with 20 ml of methylene chloride. The extract was dried under vacuo. The title compound was crystallized from a methanol-water solution (1/100, fraction 40-60°) in 25% yield.

The resulting product has the following characteristics:
(a) Melting point of 180° C.
(b) Thin layer chromatography in n-butanol/acetic acid/water (4/1/4) was carried out and the Rf value was Rf=0.66. The Rf value of the reactant, N-acetyl β,β-dimethyl cysteine is 0.88.
(c) Nuclear Magnetic Resonance (NMR) in deutarated acetone (D₆)
   4.79, 1H, d, J=6.0
   4.17, 2H, q, J=7.0
   2.81,1H, d, J=6.0
   1.98, 3H, S
   1.44, 6H, S
   1.27, 3H

### Example 3

### Synthesis of N-Acetyl Glutathione Amide (Compound I)

Ammonia gas was bubbled through absolute dry ethanol at −70° C. (dry ice with acetone), for 10 minutes. N-acetyl glutathione ethyl ester (compound G), 350 mg (1 mmol) was added to the cooled ethanol/ammonia solution and ammonia was continued to bubble through the solution for additional 10 minutes. Then, the solution was corked and was left at room temperature. After 16 hours, the flask was opened and access of ammonia and the ethanol were evaporated under reduced pressure. The product was lyophilized. The yield was 84%.

The resulting product has the following characteristics:
(a) Thin layer chromatography in n-butanol/acetic acid/water (4/1/4) was carried out and the Rf value was Rf=0.71.

### Example 4

### Synthesis of N-Acetyl Cysteine Amide (Compound J)

Ammonia gas was bubbled through absolute dry ethanol at −70° C. (dry ice with acetone), for 10 minutes. N-acetyl cysteine ethyl ester (compound A), 163 mg (1 mmol) was added to the cooled ethanol/ammonia solution and ammonia was continued to bubble through the solution for additional 10 minutes. Then, the solution was corked and was left at room temperature. After 16 hours, the flask was opened and access of ammonia and the ethanol were evaporated under reduced pressure. The product was lyophilized. The yield was 98%.

The resulting product has the following characteristics:
(a) Thin layer chromatography in n-butanol/acetic acid/water (4/1/4) was carried out and the Rf value was Rf=0.70. The Rf value of the reactant, N-acetyl cysteine ethyl ester is 0.91.

Alternatively, a solution of 20% piperidine (4 ml) in 16 ml DMF was added to Fmoc Rink amide AM resin (2 gram; 1.1 mmole amide) and the reaction was allowed to proceed for 30 minutes. Ac—S-trityl cysteine (1.3 gram, 3.3 mmole) was added with TBTU (1.06 gram) followed by diisopropyl ethyl amine (1.12 ml). The reaction was carried out for 2 hours. The resin was washed with methylene chloride (×6), and then a mixture of 1 ml silan/0.5 ml water/19 ml of TFA was added. After 1 hour the resin was filtered washed with TFA and solvents evaporated. The product was dissolved in water and extracted with methylene chloride. The aqueous solution was thereafter lyophyllized.

The resulting product has the following characteristics:
(a) Nuclear Magnetic Resonance (NMR)
   4.45 t,1,j=6.96 Hz
   2.81 ABX system, J_{AB}=12.69, J_{AX}+J_{BX}=12.45 HZ
   2.00 s 3 Hz

### Example 5

### Synthesis of N-Acetyl β,β Dimethyl Cysteine Amide (Compound K)

Ammonia gas was bubbled through absolute dry ethanol at −70° C. (dry ice with acetone), for 10 minutes. N-acetyl β,β dimethyl cysteine ethyl ester (compound B), 194 mg (1 mmol) was added to the cooled ethanol/ammonia solution and ammonia was continued to bubble through the solution for additional 10 minutes. Then, the solution was corked and was left at room temperature. After 16 hours, the flask was opened and access of ammonia and the ethanol were evaporated under reduced pressure. The product was lyophilized. The yield was 90%.

The resulting product has the following characteristics:
(a) Thin layer chromatography in n-butanol/acetic acid/water (4/1/4) was carried out and the Rf value was Rf=0.50. The Rf value of the reactant, N-acetyl β,β dimethyl cysteine ethyl ester is 0.66.

### Example 6

### Synthesis of N-Acetyl Cysteine Glycine Amide (Compound L)

A solution of 20% piperidine (4 ml) in 16 ml DMF was added to Fmoc Rink glycine amide AM resin (1.1 mmole amide) and the reaction was allowed to proceed for 30 minutes. Ac—S-trityl cysteine (1.3 gram, 3.3 mmole) was then added with TBTU (1.06 gram) followed by diisopropyl ethyl amine (1.12 ml). The reaction was carried out for 2 hours. The resin was washed with methylene chloride (×6), and then a mixture of 1 ml silan/0.5 ml water/19 ml TFA was added. After 1 hour the resin was filtered washed with TFA and solvents were evaporated. The product was dissolved in water and extracted with methylene chloride. The aqueous solution was lyophilized.

### Example 7

### In Vitro Extracellular Antioxidation by Compounds A-D

Compounds A-D were assayed in vitro for their extracellular antioxidant activities. The assays were carried out with PC12 cells (Offen et al., 1996) subjected to a high dose of dopamine which confers oxidative stress to these cells by forming oxidation products during its oxidation in the growth medium, i.e., extracellularly.

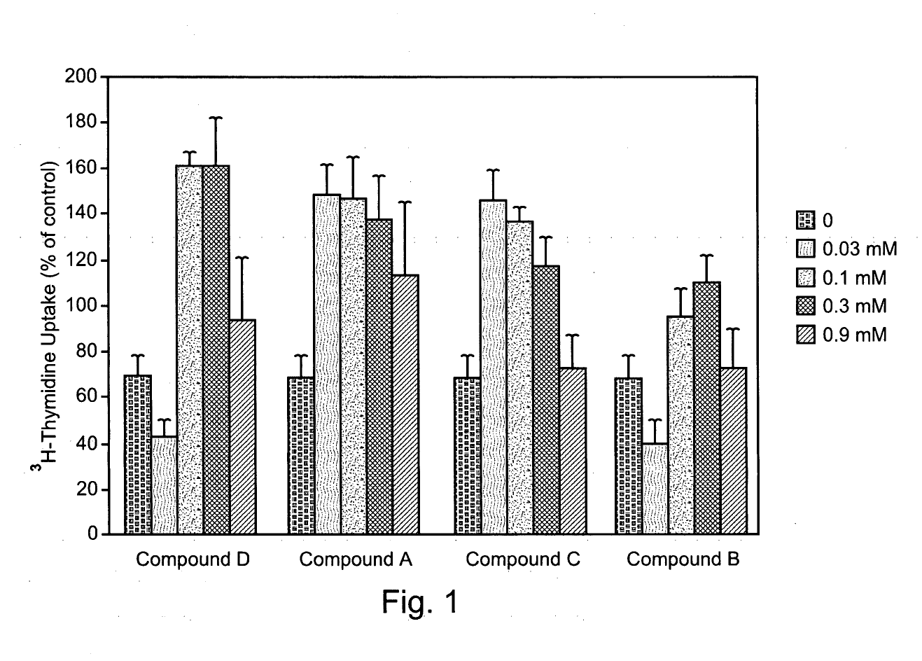

With reference now to FIG. 1. To this end, PC12 cells were subjected to high concentration of dopamine (0.5 mM) for 24 hours in the presence of increasing concentrations (0 mM, 0.03 mM, 0.1 mM, 0.3 mM and 0.9 mM) of the various compounds A-D. [³H]-thymidine was added to the cells (1 μCi/100,000 cells) six hours before the end of the 24 hours period. Due to the high lipophylicity of compounds A-D, the compounds were first dissolved in dimethyl sulfoxide (DMSO) and then in water and were applied to the cells in a final concentration of 3% DMSO. The effect of 3% DMSO on the cells was tested separately and the values presented in FIG. 1 are after the appropriate corrections.

[³H]-thymidine uptake was measured in triplicate wells containing cells pretreated with dopamine alone and dopamine with each of compounds A-D at the concentrations as indicated above. The results presented in FIG. 1 show the mean of triplicate wells taken from three independent cell batches, wherein control represent cells treated only with 3% DMSO and is defined as 100% [³H]-thymidine uptake (not shown).

Please note that all compounds A-D increased [³H]-thymidine uptake at least at one concentration value. Increase varied between Ca. 1.5 (compound B at 0.03 mM) to Ca. 2.5 (compound D at 0.03 mM and 0.1 mM). Thus, all four compounds showed high potency as protective extracellular antioxidants. Furthermore, some also reversed the basal cellular oxidation state which occurs spontaneously in control cells (not shown). Thus compounds A-D were proven useful as extracellular antioxidants.

### Example 8

### In Vitro Intracellular Antioxidation by Compounds A-D

One of the main characteristics of the oxidative effects in PC 12 cells are mimicked by 6-hydroxy-dopamine which is a false neurotransmitter taken up by the cells. Therefore, 6-hydroxy-dopamine was used as another oxidative agent and tested the protective antioxidant efficiencies of compounds A-D within the cells.

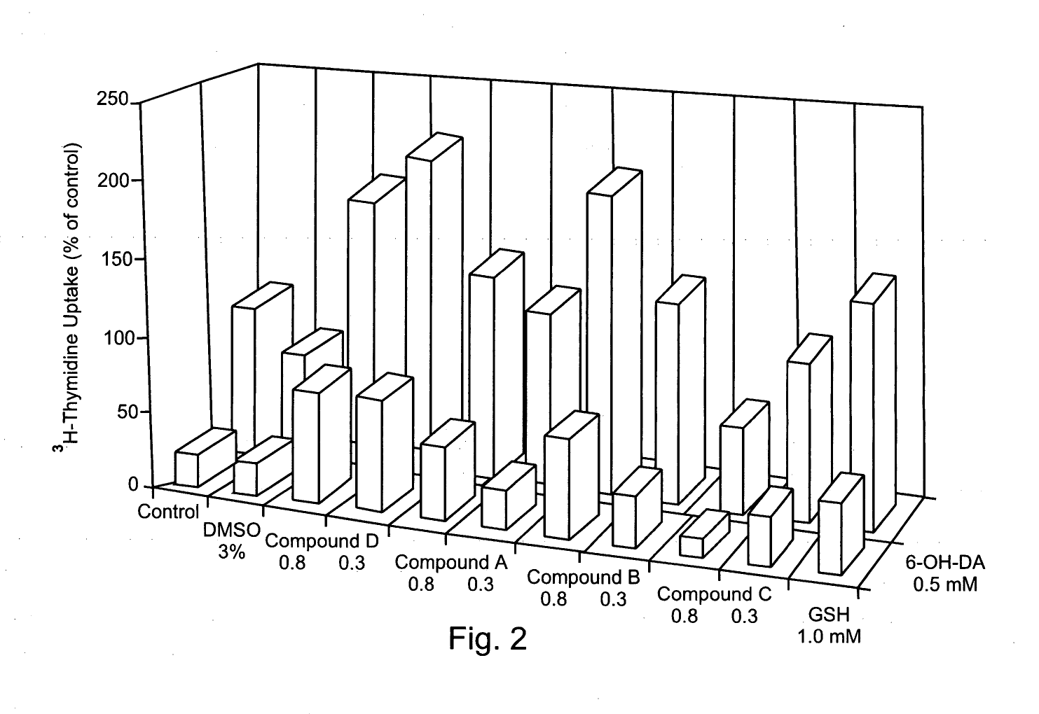

With reference now to FIG. 2. To this end, PC12 cells were subjected to high concentration (0.5 mM) of 6-hydroxy-dopamine (6-HO-DA) for 24 hour, in the presence of 0.3 mM or 0.8 mM of compounds A-D or 1 mM of reduced glutathione (GSH) a natural antioxidant, as shown in the front row of FIG. 2. A similar set of cells was treated with the same concentrations of compounds A-D and of reduced glutathione, yet without 6-hydroxy-dopamine, as shown in the back row of FIG. 2. Due to the high lipophylicity of the antioxidants used, they were first dissolved in dimethyl sulfoxide (DMSO), then in water and were applied to the cells in a final concentration of 3% DMSO. [³H]-thymidine was added to the cells (1 μCi/100,000 cells) six hours prior to the end of the 24 hour period.

The results presented in FIG. 2 are the mean of triplicate wells taken from three independent cell batches, wherein control represent un-treated cells and is defined as 100% [³H]-thymidine uptake. The effect of DMSO on the cells was tested separately as shown.

Please note that all compounds A-D increased [³H]-thymidine uptake of 6-hydroxy-dopamine treated cells, at least at one concentration value. Increase varied between Ca. 1.5 (compound C at 0.3 mM) to Ca. 3.5 (compound D at 0.3 mM and 0.8 mM). Thus, all four compounds showed high potency as protective intracellular antioxidants. Furthermore, some also reversed the basal cellular oxidation state which occurs spontaneously in cells not treated with 6-hydroxy-dopamine (FIG. 1, back row). Thus compounds A-D were proven useful as intracellular antioxidants.

### Example 9

### In Vivo Antioxidation by Compounds A-D

To demonstrate that indeed compounds A-D cross the blood brain barrier and affect oxidation state of brain cells, animals were injected with compound A and the endogenous reduced glutathione (GSH) amounts in the serum, in the corpus striatum (at the central nervous system) and/or in the whole brain were determined to evaluate compound A's antioxidation activity within the brain, as was determined by the ratio between endogenous brain (corpus striatum) GSH and endogenous serum GSH.

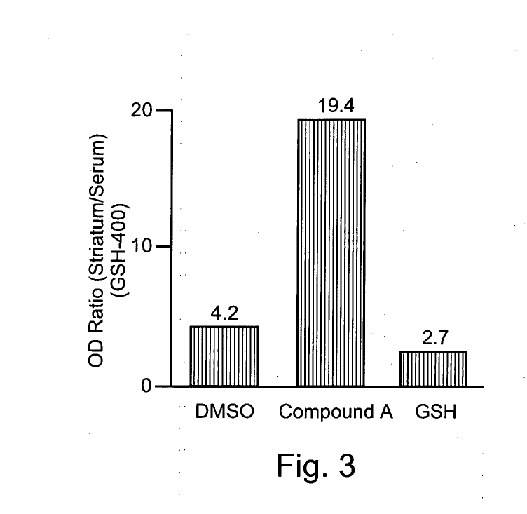

With reference now to FIG. 3. To this end, three groups of two months old Balb/c mice containing two animals in each group were injected intraperitonealy (IP) with 100-300 mg/kg body weight of compound A. Blood samples were drawn from the tail three hours post injection and then the animals were sacrificed and either the corpus striatum or the whole brain were removed and analyzed for GSH levels.

GSH levels were determined using the experimental procedures as described hereinbelow and/or the GSH-400 kit (Oxis International, Inc.).

Preparation of brain homogenates: Animals were rapidly killed and exsanguinated to remove excess blood from the brain. The brain of each animal was rinsed in a beaker containing water, lightly blotted to dry and were weighted. The striatums were transferred into a hand-homogenizer tube and each was homogenized using a constant number (e.g., 20) of up and down strokes of the hand-homogenizer pestle. Each of the homogenates was poured into a centrifuge tube and centrifuged for 10,000×g for 5 min. The supernatant was used for GSH determination as follows.

GSHAssay: For each measurement, 200 μl of sample were incubated with 20 μl DTNB [5,5′ dithio bis(2-nitrobenzoic acid)] for 1 hour in 37° C. Final absorbance was measured at 400 nm. Similar results were obtained using the GSH-400 kit.

The results shown in FIG. 3 are presented as the OD ratio of striatum/serum endogenous GSH levels. Two control mice were injected with dimethylsulfoxide (DMSO), since DMSO was used as vehicle for the injection of compound A. Exogenous GSH was also administered and used as a control for an antioxidant known not to cross the blood brain barrier.

These results demonstrate that compound A injected IP crosses the blood brain barrier and upon entry to cells at the striatum, increases the level of endogenous GSH, demonstrating its potential protection against oxidative stress.

### Example 10

### In Vivo Antioxidation by Compound J

Detection of compound j (referred to in this Example also as CEA) was established using high performance liquid chromatography (HPLC). To this end, CEA was treated with a fluorescent thiolyte reagent (monobromobimane reagent) and analyzed on an HPLC column.

Protection from oxidative stress in vitro: Neuroblastoma SHSY5Y (NB) cells were maintained in Dulbeco's Modified Eagle's Medium (DMEM), supplemented with 8% FCS and 8% horse serum, penicillin (25 μg/ml), streptomycin (25 μg/ml), 2 mM L-glutamine and 400 μg/ml G418 (Gibco/BRL). For protection experiments cells were subcultured (in 2% serum) to poly-L-lysine-coated 96-well microtiter plates (Nunc), 100 μl of 5×10⁵ cells/ml, CEA was applied to the cells in each well and 4 hours later DA, L-dopa (levodopa), 6-OHDA and MPP+ were added for 24 hours.

Survival was assayed by adding neutral red (0.34%, Sigma) to cells in DCCM-1 medium (0.1 ml/well, Bet-Haemek) and incubation for 2 hour at 37° C. The cells were then washed with cold PBS containing 10 mM MgCl₂ and the dye was dissolved in 50% ethanol in 50% Somerson buffer (70 mM sodium citrate, 30 mM citric acid, 0.1 N HCl). ELISA reader (590 nm) was used to measure the remaining color intensity.

Crossing the blood-brain-barrier: In vivo experiments were carried out on C57BL/6J mice (15 grams) injected IP with compound J. After incubation (15 min, 60 min or 4 hours) the mice were anesthetized with ether and blood samples were drawn. Then mice underwent perfusion with 50 ml of saline, injected into the right ventricle. The levels of compound I in the brain and in the plasma were detected by selective fluorescent labeling using high performance liquid chromatography (HPLC).

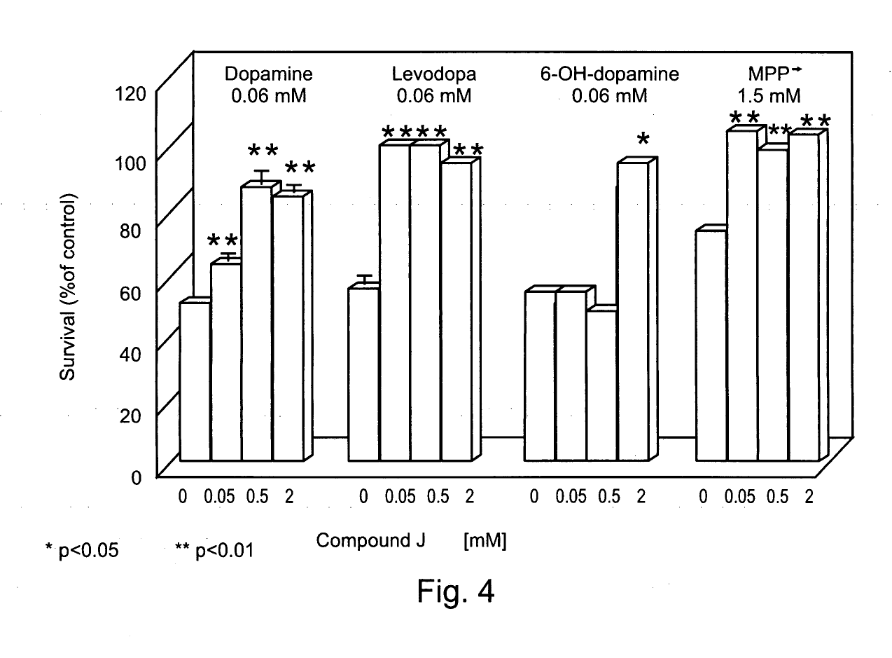

The results are shown in FIGS. 4-6.

Increasing concentrations of compound J were added to NB cells. compound J at 0.1 mM was found to protect the cells against the toxicity (>50%) of DA, L-dopa (levodopa), 6-OHDA (0.1-0.25 mM) and MPP⁺ (0.5-2 mM). Cell survival was increased up to 95% in 0.3 mM compound J as indicated by neutral red assays (FIG. 4).

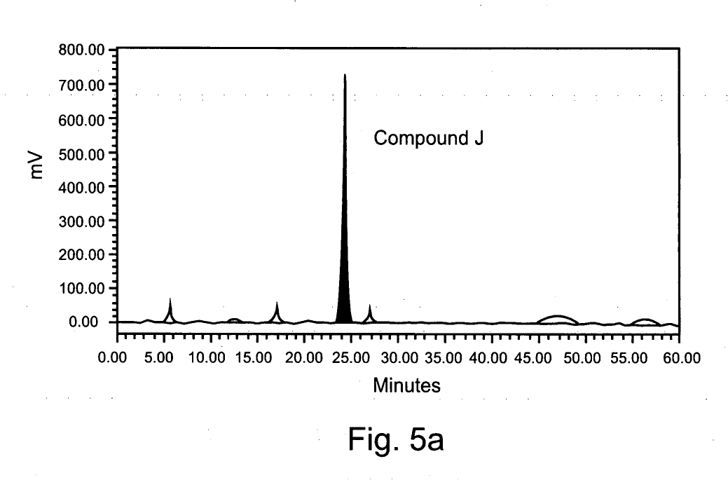

Increasing amounts of compound J were injected IP and 15 min later, mice were perfused with saline and compound J levels in the brain extracts were determined by HPLC chromatography (FIG. 5b), as compared to a control, pure, uninjected compound J (FIG. 5a).

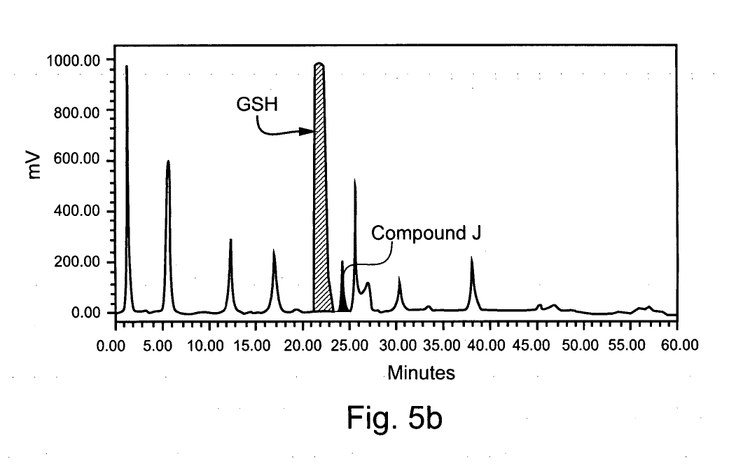

IP injection of increasing concentrations (0.25-4 mg) of compound J showed a concentration-dependent increase of compound J in the brain (FIG. 6).

As shown in FIG. 5b, GSH levels were also increased in parallel to compound J presence showing up to 46% increase over untreated animals.

These data indicate that the newly synthesized thiol-substance, compound J, effectively protects cells grown in tissue culture from oxidative stress. It crosses the BBB as shown by the combined fluorescent labeling and HPLC chromatography. Furthermore, it increases endogenous GSH levels in mice brain after IP injection.

### Example 11

### Prevention EAE in Mice

Experimental Autoimmune Encephalomyelitis (EAE) Induction:

EAE was induced in C3H.SW female mice (4-6 weeks old, Harlen, Rehovot, Israel) by immunization with the peptide encompassing amino acids 35-55 of rat myelin oligodendrocyte glycoprotein (MOG). Synthesis was carried out by the Weizmann Institute Synthesis Unit, using a solid-phase technique on a peptide synthesizer (Applied Biosystems Inc., Foster City, Calif. City).

Mice were injected subcutaneously at one site in the flank with a 200 μl emulsion containing 75 μg MOG peptide in complete Freund's adjuvant (CFA). An identical booster immunization was given at one site of the other flank one week later.

Treatment:

Mice (n=16) were injected intraperitonealy with Compound J (200 mg/kg) twice a day and in addition, were given Compound J orally in the drinking water (300 mg/kg) on day 1 of the MOG injection and for additional 35 days post injection. Control mice (n=16) were injected twice a day with 200 mg of saline for the same time period.

Clinical Score Assessment:

0=no clinical symptom's;

1=loss of tail tonicity;

2=partial hind limb paralysis;

3=complete hind limb paralysis;

4=paralysis of four limbs;

5=total paralysis;

6=death.

Experimental Results:

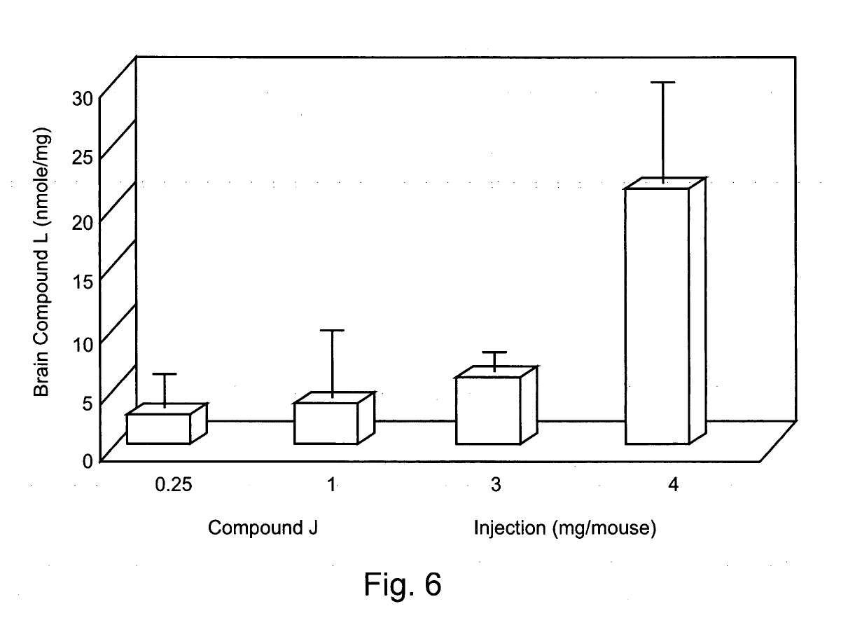

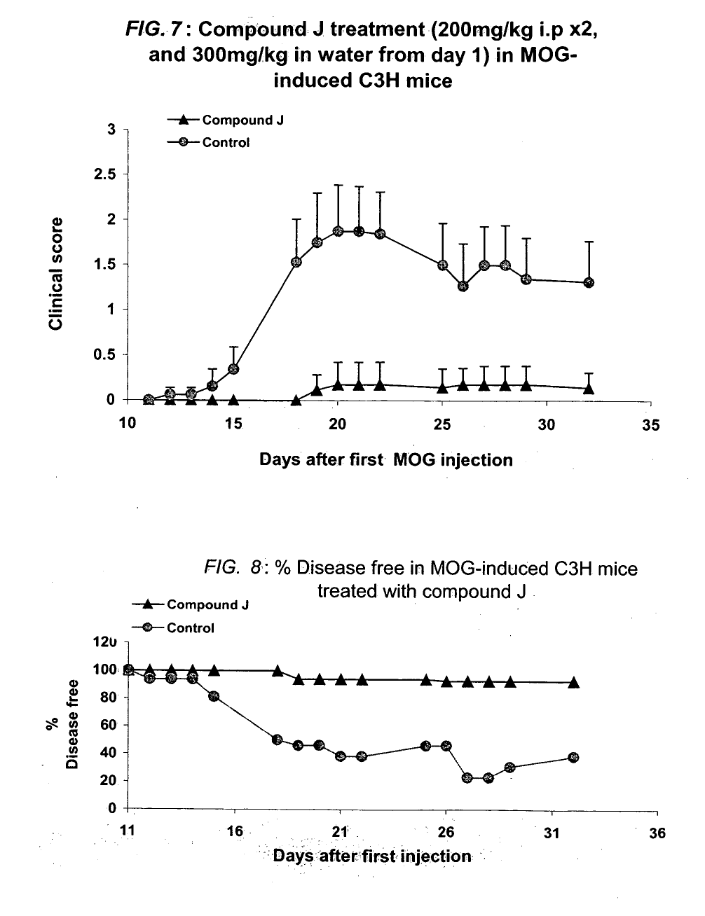

The results are shown in FIGS. 7 and 8. Following the encephalitogenic challenge, mice were observed daily and clinical manifestations of EAE were scored. Two weeks after the encephalitogenic challenge, the saline injected mice (10/16) developed severe EAE characterized by limb paralysis (mean total score of 1.7+0.2 SE) starting on day 15. In contrast, the Compound J-treated mice were significantly resistant to MOG-induced EAE. 15 out of the 16 immunized and Compound J-treated mice remained disease free with only one mouse demonstrated clinical signs (p=0.00002 using χ² values) with mean of total score of 0.1+0.1 SE (p<0.05 using Student-t-test).

Thus, Compound J treatment markedly reduced both the incidence and clinical severity of the disease.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### LIST OF REFERENCES IN ALPHABETICAL ORDER

Aschner, M., 1997. Astrocyte metallothioneins (MTs) and their neuroprotective role. Ann. N.Y. Acad. Sci. 825, pp. 334-347.
Aschner, M., Cherian, M. G., Klaassen, C. D., Palmiter, R. D., Erickson, J. C. and Bush, A. I., 1997. Metallothioneins in brain the role in the physiology and pathology. Toxicol. Appl. Pharmacol. 142, pp. 229-242.
Bartnik B L, Juurlink B H, Devon R M (2000). Mult Macrophages: their myelinotrophic or neurotoxic actions depend upon tissue oxidative stress. Mult. Scler 6 pp 37-42
Brown, D. R., Schmidt, B. & Kretzschmar, H. A. Role of microglia and host prion protein in neurotoxicity of a prion protein fragment. Nature 380, 345-347 (1996).
Butterfield D A, Lauderback C M. (2002) Lipid peroxidation and protein oxidation in Alzheimer's disease brain: potential causes and consequences involving amyloid beta-peptide-associated free radical oxidative stress (1,2). Free Radic Biol Med. 32, pp 1050-1060.
Cross, A. H., Keeling, R. M., Goorha, S., San, M., Rodi, C., Wyatts, P. S., Manning, P. T. and Misko, T. P., 1996. Inducible nitric oxide synthase gene expression and enzime activity correlate with disease activity in murine EAE. J. Neuroimmunol. 71, pp. 145-153.
Cross, A. H., Misko, T. P., Lin, R. F., Hickey, W. F., Trotter, J. L. and Tilton, R. G., 1994. Aminoguanidine, an inhibitor of inducible nitric oxide synthase, ameliorates EAE in SJL mice. J. Clin. Invest. 93, pp. 2684-2690.
Ebadi, M., Iversen, P. L., Hao, R., Cerutis, D. R., Rojas, P., Happe, H. K., Murrin, L. C. and Pfeiffer, R. F., 1995. Expression and regulation of brain metallothionein. Neurochem. Int. 27, pp. 1-22
Espejo C, Carrasco J, Hidalgo J, Penkowa M, Garcia A, Saez-Torres I, Martinez-Caceres E M Differential expression of metallothioneins in the CNS of mice with experimental autoimmune encephalomyelitis. Neuroscience 2001;105(4):1055-65
Fahn S. & Cohen, G. The oxidant stress hypothesis in Parkinson's disease: evidence supporting it. Ann Neurol. 32, 804-812 (1992).
Fenyk-Melody, J. E., Garrison, A. E., Brunnert, S. R., Weidner, J. R., Shen, F., Shelton, B. A. and Mudgett, J. S., 1998. Experimental autoimmune encephalomyelitis is exacerbated in mice lacking the NOS2 gene. J. Immunol. 160, pp. 2940-2946.
Hooper, D. C., Ohnishi, S. T., Kean, R., Numagami, Y., Dietzschold, B. and Koprowski, H., 1995. Local nitric oxide production in viral and autoimmune diseases of the central nervous system. Proc. Natl. Acad. Sci. USA 92, pp. 5312-5316
Lassmann, H., Raine, C. S., Antel, J. and Prineas, J. W., 1998. Immunopathology of multiple sclerosis: report on an International Meeting Held at the Institute of Neurology of the University of Vienna. J. Neuroimmunol. 86, pp. 213-217.
Lazo, J. S., Kondo, Y., Dellapiazza, D., Michalska, A. E., Choo, K. H. and Pitt, B. R., 1995. Enhanced sensitivity to oxidative stress in cultured embryonic cells from transgenic mice deficient in metallothionein I and II genes. J. Biol. Chem. 270, pp. 5506-5510.
Lin, R. F., Lin, T-S., Tilton, R. G. and Cross, A. H., 1993. Nitric oxide localized to spinal cords of mice with experimental allergic encephalomyelitis: an electron paramagnetic resonance study. J. Exp. Med. 178, pp. 643-648.
Martin, R., McFarland, H. F. and McFarlin, D., 1992. Immunological aspects of demyelinating diseases. Annu. Rev. Immunol. 10, pp. 153-187
Okuda, Y., Sadoka, S., Fujimura, H. and Yanahiara, T., 1995. Nitric oxide via an inducible isoform of nitric oxide synthase is a possible factor to eliminate inflammatory cells from the central nervous system of mice with EAE. J. Neuroimmunol. 73, pp. 107-116.
Olanow, C. W. A. radical hypothesis for neurodegeneration. Trends. Neurol. Soc. 16, 439-444 (1993).
Olanow, C. W. Oxidation reactions in Parkinson's disease. Neurology 40, 32-37 (1990).
Ruuls, S. R., Van Der Linden, S., Sontrop, K., Huitinga, I. and Dijkstra, C. D., 1996. Aggravation of EAE by administration of nitric oxide synthase inhibitors. Clin. Exp. Immunol. 103, pp. 467-476.
Sahrbacher, U. C., Lechner, F., Eugster, H-P, Frei, K., Lassmann, H. and Fontana, A., 1998. Mice with an inactivation of the inducible nitric oxide sinthase gene are susceptible to experimental autoimmune encephalomyelitis. Eur. J. Immunol. 28, pp. 1332-1338.
Schwarz, M. A., Lazo, J. S., Yalowich, J. L., Allen, W. P., Whitmore, M., Bergonia, H. A., Tzeng, E., Billiar, T. R., Robbins, P. D., Lancaster, J. R. and Pitt, B. R., 1995. Metallothionein protects against cytotoxic and DNA-damaging effects of NO. Proc. Natl. Acad. Sci. USA 92, pp. 44452-44456.
Shiraga, H., Pfeiffer, R. F. and Ebadi, M., 1993. The effects of 6-hydroxydopamine and oxidative stress on the level of brain metallothionein. Neurochem. Int. 23, pp. 561-566.
Steinman L, Martin R, Bernard C, Conlon P, Oksenberg J R. MULTIPLE SCLEROSIS: Deeper Understanding of Its Pathogenesis Reveals New Targets for Therapy. (2002) Annu Rev Neurosci. 25 pp 491-505.
Steinman L. (2001). Multiple sclerosis: a two stage disease. Nat. Immunol. 2, pp 762-65
Syburra, C. and Passi, S., 1999. Oxidative stress in patients with multiple sclerosis. Ukr. Biokhim. Zh. 71, pp. 112-115
Thomas, T., Thomas, G. M., McLendon, C., Sutton, T. & Mullan, M. β-Amyloid-mediated vasoactivity and vascular endothelial damage. Nature 380, 168-171 (1996).
Thornalley, P. J. and Vasak, M., 1985. Possible role for metallothionein in protection against radiation-induced oxidative stress. Kinetics and mechanism of its reaction with superoxide and hydroxyl radicals. Biochim. Biophys. Acta 827, pp. 36-44.
Tsangaris, G. T. and Tzortzatou-Stathopoulou, F., 1998. Metallothionein expression prevents apoptosis: a study with antisense phosphorothioate oligodeoxynucleotides in a human T cell line. Anticancer Res. 18, pp. 2423-2433.
Vladimirova, O., O'Connor, J., Cahill, A., Alder, H., Butonoi, C. and Kalman, B., 1998. Oxidative damage to DNA in plaques of MS brains. Mult. Scler. 4, pp. 413-418.

What is claimed is:

1. A method of treating multiple sclerosis, the method comprising administering to a subject in need thereof a therapeutically effective amount of a compound, said compound having:
(a) a combination of molecular weight and membrane miscibility properties for permitting said compound to cross the blood brain barrier of the organism;
(b) a readily oxidizable chemical group for exerting antioxidation properties; and
(c) a chemical make-up for permitting said compound or its intracellular derivative to accumulate within the cytoplasm of cells.

2. The method of claim 1, wherein said compound is selected from the group consisting of N-acetyl cysteine ethyl ester (compound A), β,β-dimethyl cysteine ethyl ester (compound B), N-acetyl-β,β-dimethyl cysteine (compound C), Glutathione ethyl ester (compound D), N-acetyl glutathione ethyl ester (compound E), N-acetyl glutathione (compound F), N-acetyl α-glutamyl ethyl ester cysteinyl glycyl ethyl ester (compound G) N-acetyl α-glutamyl ethyl ester cysteinyl glycyl (compound H), N-acetyl glutathione amide (compound I), N-acetyl cysteine amide (compound J), N-acetyl β,β dimethyl cysteine amide (compound K) and N-acetyl cysteine glycine amide (compound L).

3. The method of claim 1, wherein said readily oxidizable chemical group is a sulfhydryl group.

4. The method of claim 1, wherein said chemical make-up is selected having an ester moiety which is removable by hydrolysis imposed by intracellular esterases.

5. The method of claim 4, wherein said ester moiety is selected from the group consisting of alkyl ester and aryl ester.

6. The method of claim 5, wherein said alkyl and aryl esters are selected from the group consisting of methyl ester, ethyl ester, hydroxyethyl ester, t-butyl ester, cholesteryl ester, isopropyl ester and glyceryl ester.

7. A method of therapeutically or prophylactically treating a subject against multiple sclerosis, the method comprising administering to the individual a therapeutically or prophylactically effective amount of an antioxidant compound, said antioxidant compound having:
(a) a combination of molecular weight and membrane miscibility properties for permitting said compound to cross the blood brain barrier of the individual;
(b) a readily oxidizable chemical group for exerting antioxidation properties; and
(c) a chemical make-up for permitting said compound or its intracellular derivative to accumulate within brain cells of the individual.

8. The method of claim 7, wherein said compound is selected from the group consisting of N-acetyl cysteine ethyl ester (compound A), β,β-dimethyl cysteine ethyl ester (compound B), N-acetyl-β,β-dimethyl cysteine (compound C), Glutathione ethyl ester (compound D), N-acetyl glutathione ethyl ester (compound E), N-acetyl glutathione (compound F), N-acetyl α-glutamyl ethyl ester cysteinyl glycyl ethyl ester (compound G) N-acetyl α-glutamyl ethyl ester cysteinyl glycyl (compound H), N-acetyl glutathione amide (compound I), N-acetyl cysteine amide (compound J), N-acetyl β,β dimethyl cysteine amide (compound K) and N-acetyl cysteine glycine amide (compound L).

9. The method of claim 7, wherein said readily oxidizable chemical group is a sulfhydril group.

10. The method of claim 7, wherein said chemical make-up is selected having an ester moiety which is removable by hydrolysis imposed by intracellular esterases.

11. The method of claim 10, wherein said ester moiety is selected from the group consisting of alkyl ester and aryl ester.

12. The method of claim 11, wherein said alkyl and aryl esters are selected from the group consisting of methyl ester, ethyl ester, hydroxyethyl ester, t-butyl ester, cholesteryl ester, isopropyl ester and glyceryl ester.

13. A pharmaceutical composition for therapeutically or prophylactically treating a subject against multiple sclerosis, the composition comprising a pharmaceutically acceptable carrier and, as an active ingredient, a therapeutically or prophylactically effective amount of an antioxidant compound, said compound having:
(a) a combination of molecular weight and membrane miscibility properties for permitting said compound to cross the blood brain barrier of the individual;
(b) a readily oxidizable chemical group for exerting antioxidation properties; and
(c) a chemical make-up for permitting said compound or its intracellular derivative to accumulate within brain cells of the individual.

14. The pharmaceutical composition of claim 13, wherein said compound is selected from the group consisting of N-acetyl cysteine ethyl ester (compound A), β,β-dimethyl cysteine ethyl ester (compound B), N-acetyl-β,β-dimethyl cysteine (compound C), Glutathione ethyl ester (compound D), N-acetyl glutathione ethyl ester (compound E), N-acetyl glutathione (compound F), N-acetyl α-glutamyl ethyl ester cysteinyl glycyl ethyl ester (compound G) N-acetyl α-glutamyl ethyl ester cysteinyl glycyl (compound H), N-acetyl glutathione amide (compound I), N-acetyl cysteine amide (compound J), N-acetyl β,β dimethyl cysteine amide (compound K) and N-acetyl cysteine glycine amide (compound L).

15. The pharmaceutical composition of claim 13, wherein said pharmaceutically acceptable carrier is selected from the group consisting of a thickener, a buffer, a diluent, a surface active agent and a preservatives.

16. The pharmaceutical composition of claim 13, wherein said readily oxidizable chemical group is a sulfhydril group.

17. The pharmaceutical composition of claim 13, wherein said chemical make-up is selected having an ester moiety which is removable by hydrolysis imposed by intracellular esterases.

18. The pharmaceutical composition of claim 17, wherein said ester moiety is selected from the group consisting of alkyl ester and aryl ester.

19. The pharmaceutical composition of claim 18, wherein said alkyl and aryl esters are selected from the group consisting of methyl ester, ethyl ester, hydroxyethyl ester, t-butyl ester, cholesteryl ester, isopropyl ester and glyceryl ester.
